(19) United States
(12) Patent Application Publication
(10) Pub. No.: US 2018/0015218 A1
(43) Pub. Date: Jan. 18, 2018

(54) MEDICAL DEVICE CONTROL

(71) Applicant: Smiths Medical ASD, Inc., Plymouth, MN (US)

(72) Inventor: Michael WELSCH, Stillwater, MN (US)

(73) Assignee: Smiths Medical ASD, Inc., Plymouth, MN (US)

(21) Appl. No.: 15/538,112

(22) PCT Filed: Jan. 7, 2016
(86) PCT No.: PCT/US2016/012481

§ 371 Date: Jun. 20, 2017

Related U.S. Application Data
(60) Provisional application No. 62/105,832, filed on Jan. 21, 2015.

(51) Int. Cl. A61M 5/142; A61M 5/172

(52) U.S. Cl. CPC A61M 5/142; A61M 5/172; A61M 2205/276; A61M 2205/6063; A61M 2205/3576

(57) ABSTRACT

A method for pairing a portable device with a medical device can include displaying an association code on a dynamic display of the medical device and determining whether the portable device is positioned relative to the medical device within a predetermined range of positioning parameters. If the portable device is positioned relative to the medical device within the predetermined range of positioning parameters, the method can include optically reading the association code from the dynamic display of the medical device with the portable device. Only if the portable device is positioned relative to the medical device within the predetermined range of positioning parameters, and the association code is optically read from the dynamic display of the medical device with the portable device, then the method can include pairing the portable and medical devices such that the medical device responds to commands sent from the portable device to the medical device.

### TECHNICAL FIELD

This disclosure relates to medical devices, and more particularly, to systems and methods for controlling medical devices.

### BACKGROUND

Many medical devices, such as infusion pumps, are microprocessor controlled. In some cases, it may be desirable to control a medical device remotely, or at a distance from the medical device, without requiring direct physical access to the medical device. Such embodiments are described in this disclosure as being operated under “remote control”. In applications where microprocessor controls are used (whether in medical or other technologies), the present state of electronics and information technology may make it relatively straightforward, in some aspects, to implement remote control systems. However, it is of utmost importance to minimize the possibility of error in the operation of medical devices. While it might be tolerable, for example, for a household remote control unit to turn off a DVD player mistakenly rather than a television, the consequences of mis-controlling a medical device can be severe. It would therefore be desirable to provide improved systems and methods for remotely controlling medical devices while reducing possible sources of error and minimizing the chances of creating hazards when providing such technologies.

### SUMMARY

This disclosure relates to medical devices, and more particularly, to systems and methods for controlling medical devices.

In an illustrative but non-limiting example, the disclosure provides a method for pairing a portable device with a medical device. The method can include displaying an association code on a dynamic display of the medical device and determining whether the portable device is positioned relative to the medical device within a predetermined range of positioning parameters. If the portable device is positioned relative to the medical device within the predetermined range of positioning parameters, the method can include optically reading the association code from the dynamic display of the medical device with the portable device. Only if: (a) the portable device is positioned relative to the medical device within the predetermined range of positioning parameters, and (b) the association code is optically read from the dynamic display of the medical device with the portable device, then the method can include pairing the portable device and the medical device such that the medical device responds to commands sent from the portable device to the medical device. If (c) the portable device is not positioned relative to the medical device within the predetermined range of positioning parameters, then the method can include not pairing the portable device and the medical device. The method can further include the medical device executing a medical function in response to a command sent from the portable device. Examples of medical functions that the medical device could execute include delivering a medicament to a patient or measuring a vital sign of a patient.

In some cases, the predetermined range of positioning parameters can be defined independently of particular hardware capabilities of the portable device. In some cases, the predetermined range of positioning parameters can define positioning a camera of the portable device within 50 cm of the association code displayed on the medical device and within 5 cm of an axis centered on the association code and normal to the association code.

In some cases, pairing the portable device and the medical device can include receiving confirmation from a user to make the pairing. Receiving confirmation from a user in some cases does not require physical contact with the medical device.

In some cases, the method can include communicating from the portable device information related to the association code read by the portable device, via a wireless communication mode. Further, the method can include a processor external to the portable device and the medical device receiving the information related to the association code read by the portable device. Pairing the portable device and the medical device includes the external processor, in response to receiving the information related to the association code, commanding pairing of the portable device and the medical device.

The method can further include pairing the portable device and a second medical device such that the second medical device responds to commands sent from the portable device to the second medical device. In such a case with both a (first) medical device and a second medical device, the method can further include rendering on a display of the portable device a virtual representation of the medical device and the second medical device that emulates a real-world spatial relationship between the medical device and the second medical device.

In some cases, the method can further include optically reading, with the portable device, an identification code that identifies the medical device, and displaying the association code on the dynamic display of the medical device can be performed in response to optically reading the identification code.

In some cases, if the portable device is not positioned relative to the medical device within the predetermined range of positioning parameters, then in the method the association code is not optically read from the dynamic display of the medical device with the portable device.

In another illustrative but non-limiting example, the disclosure provides another method for pairing a portable device with a medical device. The method can include optically reading, with the portable device, an identification code that identifies the medical device; commanding the medical device to display an association code on a dynamic display of the medical device; and determining whether the portable device is positioned relative to the medical device within a predetermined range of positioning parameters. If the portable device is positioned relative to the medical device within the predetermined range of positioning parameters, the method can include optically reading the association code from the dynamic display of the medical device with the portable device. Only if: (a) the portable device is positioned relative to the medical device within the predetermined range of positioning parameters, and (b) the association code is optically read from the dynamic display of the medical device with the portable device, then the method can include pairing the portable device and the medical device such that the medical device responds to commands sent from the portable device to the medical device. If (c) the portable device is not positioned relative to the medical device within the predetermined range of positioning parameters, then the method can include not pairing the portable device and the medical device. In some instances of the method, commanding the medical device to display an association code on a dynamic display of the medical device can be performed in response to reading the identification code. In some cases, commanding the medical device to display an association code can include a processor of the portable device commanding the medical device to display the association code.

In some cases the identification code can be displayed on the medical device. In some cases, the association code can include information particular to a current status of the medical device.

In some cases, the method can further include communicating from the portable device information related to the identification code read by the portable device, via a wireless communication mode. Further, commanding the medical device to display an association code can include a processor external to the portable device receiving the information related to the identification code read by the portable device, and in response, the processor commanding the medical device to display the association code.

In some cases, the method can further include communicating from the portable device information related to the association code read by the portable device via a wireless communication mode. Furthermore, pairing the portable device and the medical device can include a processor external to the portable device receiving the information related to the association code read by the portable device, and in response, the processor pairing the portable device and the medical device.

In yet another illustrative but non-limiting example, the disclosure provides a medical device system that can include a medical device and a portable device. The medical device can be configured to provide at least one of a therapeutic or a patient monitoring function, and can include a display capable of displaying a machine-readable code, a communication interface, and a controller operatively coupled to the display and the communication interface. The a portable device can include a user interface configurable to present virtual controls for the medical device to a user, an optical imaging device, a wireless communication interface, and a controller operatively coupled to the user interface, the optical imaging device, and the wireless communication interface. In the method, the controller of the medical device can be programmed and configured to display a machine-readable association code on the display of a medical device; and pair with the portable device either under the command of the controller of the portable device or an external processor, or after the controller of the medical device has determined that predetermined conditions for entering paired communication with the portable device have been met. When paired, the medical device can respond to commands sent from the portable device to the medical device. In the method, the controller of the portable device can be programmed and configured to determine whether the portable device is positioned relative to the medical device within a predetermined range of positioning parameters, and if the controller determines that the portable device is positioned relative to the medical device within the predetermined range of positioning parameters: read the association code from the dynamic display of the medical device with the optical imaging device of the portable device, and communicate, via the wireless communication interface, information related to the association code read by the optical imaging device.

In some cases, the controller of the portable device can be further programmed and configured to: (1) if the association code is read from the dynamic display of the medical device, determine whether predetermined conditions for entering paired communication with the medical device have been met, including considering information related to the association code; and (2) if predetermined conditions for entering paired communication with the medical device are determined to have been met, communicate, via the wireless communication interface, a command to the controller of the medical device to pair with the portable device.

In some cases, the system can further an external computing system separate from, and communicatively coupled with, the medical device and the portable device. The external computing system can be programmed and configured to: receive from the portable device information related to the association code read by the optical imaging device; determine whether predetermined conditions for entering paired communication between the medical device and the portable device have been met; and if predetermined conditions for entering paired communication between the medical device and the portable device are determined to have been met, communicate commands to the controller of the medical device and the controller of the portable device to pair.

The above summary is not intended to describe each and every example or every implementation of the disclosure. The Description that follows more particularly exemplifies various illustrative embodiments.

### BRIEF DESCRIPTION OF THE FIGURES

The following description should be read with reference to the drawings. The drawings, which are not necessarily to scale, depict examples and are not intended to limit the scope of the disclosure. The disclosure may be more completely understood in consideration of the following description with respect to various examples in connection with the accompanying drawings, in which:

FIG. 1 is a schematic illustration of a medical system that includes a plurality of medical devices, a computing or information system, and a portable device;

FIG. 2 is a schematic illustration of the portable device of FIG. 1, a medical device such as any of the medical devices of FIG. 1, and the external information system of FIG. 1;

FIG. 3 is a flow diagram of an illustrative example of a method for pairing a portable device with a medical device;

FIG. 4 is a schematic illustration of relative positioning between a medical device such as any of the medical devices of FIG. 1, and the portable device of FIG. 1;

FIG. 5 is a flow diagram of another illustrative example of a method for pairing a portable device with a medical device; and

FIG. 6 is a schematic representation of the portable device of FIG. 1 with, on its display, a simulated virtual representation of the medical devices of FIG. 1 and a simulated virtual control panel for one of the medical devices.

### DESCRIPTION

The following description should be read with reference to the drawings, in which like elements in different drawings may be numbered in like fashion. The drawings, which are not necessarily to scale, depict selected examples and are not intended to limit the scope of the disclosure. Although examples of construction, dimensions, and materials may be illustrated for the various elements, those skilled in the art will recognize that many of the examples provided have suitable alternatives that may be utilized.

In the present disclosure, the following conditional expressions should be understood to agree with conventional formal logic, as summarized thus:

“A if B” means that B is a sufficient (but not necessarily necessary) condition for A. “A if B” can be expressed alternately as “if B, (then) A”.

“A only if B” means that B is a necessary (but not necessarily sufficient) condition for A. “A only if B” can be expressed alternately as “only if B, (then) A”.

“A if and only if B” means that B is a necessary and sufficient condition for A. “A if and only if B” can be expressed alternately as “if and only if B, (then) A”.

Many medical devices have user interface elements physically integral to the devices, such as buttons, knobs, switches, and the like located on, in, or with device housings, and/or information displays such as lights, gauges, meters, segmented and/or pixelated displays, and the like placed similarly. User interface elements that are physically integral to a medical device can be intrinsically, inherently, unambiguously, and obviously associated with the device. With such an association, it is a reasonable expectation that a user interacting with physically integrated user interface elements will not be confused or uncertain about the device with which the user is interacting.

In some situations, it may be desirable to interact with a user interface for a medical device at a location spatially remote or physically at a distance from the main, major or primary component(s) of the medical device. This could be desired for any number of reasons, such as a want or need for a centralized control system for multiple medical devices, for infection control, for providing a richer user interface when not constrained to the main part(s) of the device, or any other appropriate reason. In medical device applications where microprocessor controls are used, modern technology can make it relatively straightforward, in some aspects, to implement remote control systems. Portable computing devices such as smartphones, tablet or pad computers, and the like can be ubiquitous in many modern settings, including hospitals, other medical caregiving facilities, and home environments. Many such devices can be well-equipped to provide remote-control functionalities, with robust communication capabilities, rich user-interface hardware such as touchscreens, and operating systems that can capably run remote control application software. In light of these technological advances, it may be considered desirable to provide the capability to use portable devices to remotely control medical devices. (Note that while many medical devices may be considered portable and many include computerized features, in the present disclosure the term “portable device” is used in reference to portable computing devices such as smartphones, tablet computers, etc. The use of the term “portable device” in reference to these types of devices does not negate the portability of any medical or other device not explicitly described as “portable”.) However, without the kind of direct physical link between user interface and medical device that inherently exists when user interface elements are physically integral to the medical device, employing a remote control user interface may introduce the hazard of remotely controlling the wrong or unintended medical device.

The present disclosure is therefore directed toward systems and methods to reduce the possibility that a portable device is misconfigured to remotely control a medical device other than one that it is intended to remotely control. In some aspects, the present disclosure provides systems and methods that can aid in reliably pairing portable devices with medical devices as intended by a user, and to reduce the risk that a portable device is inadvertently or accidentally paired to a device or devices other than intended by the user. “Pairing” can refer to a process that concerns the establishment of a networking and/or operative linkage between computing devices. (While the term “pairing” may commonly be associated with the Bluetooth wireless technology standard and other standards, in the present disclosure it is not limited to any specific technology standard or to the particular pairing protocols of any standard.) It is envisioned that the systems and methods can be implemented with portable devices that can be relatively standard, commercial, and/or off-the-shelf devices, and that can be mass-market consumer devices, such as smartphones, tablet or pad computers, personal digital assistants, and any other suitable devices that include suitable hardware components and a suitable operating system capable of running remote control application software.

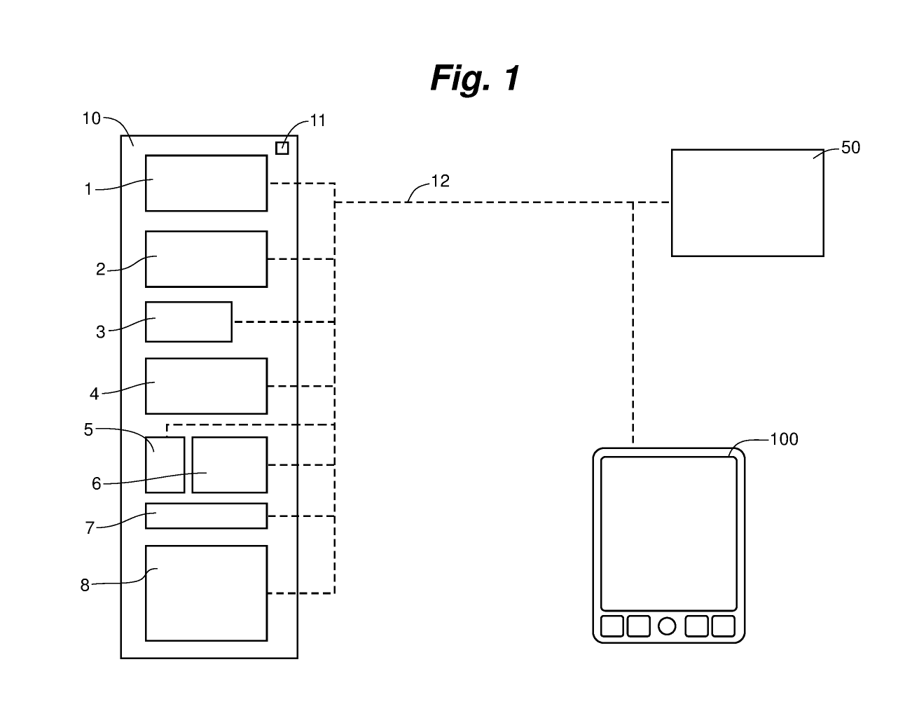

FIG. 1 is a schematic illustration of a medical system that includes a plurality of medical devices 1-8, a computing or information system 50, and a portable device 100. A quantity of eight medical devices 1-8 is illustrated, but the number depicted is arbitrary. The systems and methods of the present disclosure may include and be practiced with any suitable number of medical devices, including only a single device. The dissimilar shapes of the boxes schematically representing medical devices 1-8 may suggest a heterogeneous collection of devices, but the systems and methods of the present disclosure can include and be practiced with any suitable combination of similar and dissimilar devices. In some examples, some or all of medical devices 1-8 are infusion pumps.

Medical devices 1-8 can be communicatively connected, via any appropriate communication infrastructure(s) 12, to each other, to a computing or other information system 50, and/or to portable device 100, in any appropriate combination(s) and manner. The dashed lines representing communication infrastructure(s) 12 of FIG. 1 are merely schematic and should not necessarily be interpreted as limiting the communication infrastructure(s) of the present disclosure to any particular communication infrastructure topology, technology, layers, protocols, or any other aspect(s) of how communication can be implemented. Furthermore, illustrated connections (via the dashed lines representing communication infrastructure(s) 12) should not necessarily be interpreted as being alike between all devices and systems, and should not necessarily be assumed to be bidirectional. For example, medical devices 1-8 could each be communicatively connected to information system 50 via a wired connection (such as Ethernet or any other suitable protocol), an unwired connection (such as WiFi or any other suitable protocol), or both, whereas portable device 100 could be connected to information system 50 and/or any of medical devices 1-8 via a wireless connection only. Any appropriate communication devices such as routers or repeaters can be employed in communication infrastructure(s) 12. Medical devices 1-8, portable device 100, and/or information system 50 can in some cases communicate via communication infrastructure devices such as routers, but in some embodiments can communicate directly with each other in peer-to-peer modes. In some embodiments, one or more of medical devices 1-8, portable device 100, and/or information system 50 can provide an infrastructure function, such as router. In some arrangements, communication infrastructure 12 can include any suitable combinations of peer-to-peer and network-based communication. In some embodiments, portable device 100 could communicate directly (for example, via a short-range radio frequency protocol such as Bluetooth) with one of medical devices 1-8, and the medical device could relay information from the portable device to information system 50. In some embodiments, portable device 100 could communicate directly (for example, via an ad-hoc WiFi connection) with information system 50, and the information system could relay information from the portable device to one or more of medical devices 1-8. In some embodiments, secure communication protocols, such as secure sockets layer (SSL) and/or transport layer security (TLS) can be employed for any of the various communication links between portable device 100, medical devices 1-8, and/or information system 50 that may be employed in systems or methods of the present disclosure. These are just some examples. In summary, the systems and methods of the present disclosure can include and be implemented by and with any communication infrastructure 12 suitable to realize the functions of the systems and methods, and should not be considered limited to any particular communication architecture unless specifically described as such.

Although information system 50 is depicted in FIG. 1 as being external to medical device 1-8, it is to be appreciated and understood that such computing or information system or systems could reside in any location, such as externally or remotely in a hospital's IT infrastructure, or physically adjacent to or near the medical devices, or even internally within one or more of the devices (other than within one of the devices relative to which the information system 50 is described as being “external”). It is further to be appreciated and understood that information system 50 should not necessarily be considered to be limited to a single physical device (although in some embodiments it may be), but rather, any suitable system or arrangement of one or several components can be used to provide an information processing capability, such as one or more processes being executed by a networked system of servers.

Medical devices 1-8 can be mounted, attached, and/or otherwise connected with or to a support structure 10, such as an equipment rack or pole, but this is not necessary. The support structure 10 can mechanically hold the medical devices in fixed physical relationships with respect to each other, and can, in some instances, provide other functions such as electrical power and/or communication cabling. In the latter case, the support structure 10 could thereby provide a portion of communication infrastructure 12. In some embodiments the support structure 10 could include networking hardware (such as a router) with ports for any or all devices mounted thereto. Medical devices mounted to support structure 10 and connected (whether physically, as wired, or wirelessly) to the networking hardware associated with the support structure could be automatically associated with each other by way of connection to the networking hardware.

In some embodiments, one or more of the medical devices can be a portable medical device, configured to move with a patient (possibly even carried by a patient) as the patient moves about, and may be referred to as an “ambulatory” medical device. Some example systems of the present disclosure include only a single ambulatory medical device.

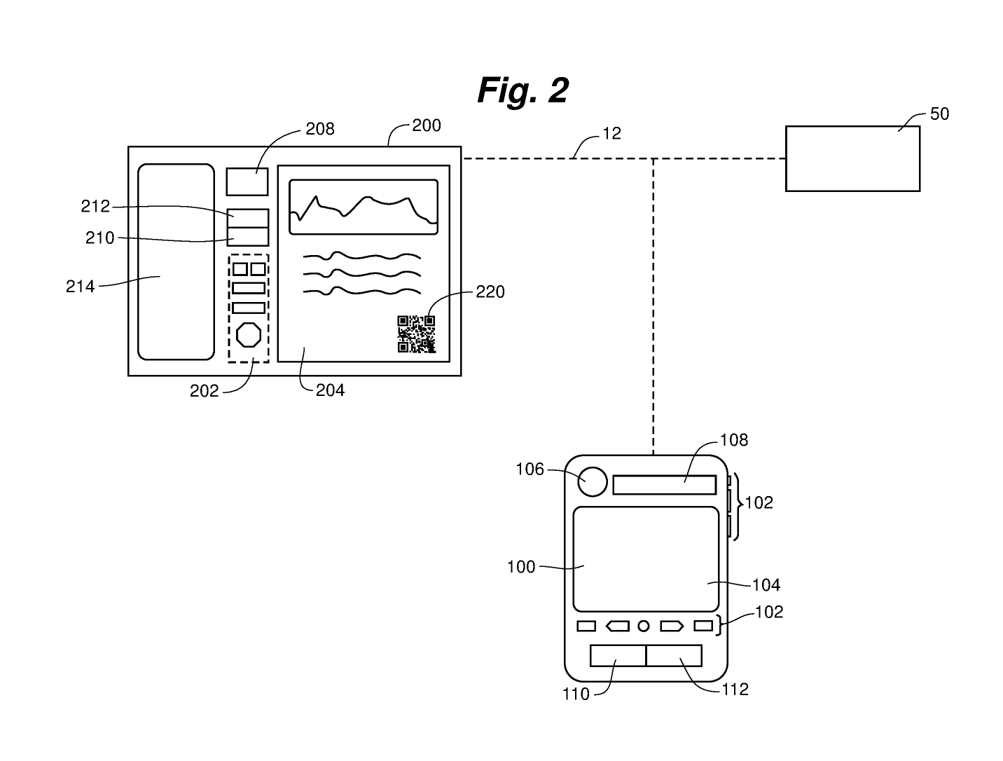

FIG. 2 is a schematic illustration of portable device 100 of FIG. 1, a medical device 200 that can be any suitable medical device, such as any of medical devices 1-8 of FIG. 1, and external information system 50. Portable device 100 can be any suitable device. (Reference numeral 100 is used throughout the figures of this application in reference to a portable device, and features of a portable device 100 described in relation to any particular figure should not necessarily be considered to be limited only to the configuration of that figure. All illustrations of portable device 100 in this application are merely schematic and should not be considered limiting. Differences in the appearance of portable device 100 between drawings do not necessarily represent substantive feature differences between embodiments of portable devices.) Portable device 100 can be a widely- and/or commercially-available multi-purpose device such as a smartphone, tablet or pad computer, personal digital assistant, or the like. In other embodiments, it can be a device specifically built for the purpose of being a remote control device for medical devices. Portable device 100 can include a user interface, which can include hardware controls 102 such as buttons and/or dials and the like, and a display 104. The user interface can be configurable to present to a user virtual controls for a medical device that is paired with portable device 100. Display 104 can employ any suitable display technology such as liquid crystal display (LCD), organic light-emitting diode (OLED), electronic paper display (EPD), and the like. Display 104 can be a touchscreen display structured with any suitable touch-sensing technology, such as capacitive, resistive, and so on. When display 104 is configured as a touchscreen display it can be suitable for providing a virtual control panel for a medical device with which the portable device 100 is paired. Such a virtual control panel can be configured to emulate the look and feel of the actual physical hardware of the medical device, but is not limited to such an emulation. A virtual control panel could provide alternative or enhanced user interface features as compared to the hardware of the medical device.

Portable device 100 can include an optical imaging device 106 (such as, but not limited to, a digital camera module), a wireless communication interface 108, a memory 110, and a controller 112. The controller 112 can be operatively coupled to any or all of the user interface (for example, hardware controls 102 and/or display 104), the optical imaging device 106, the wireless communication interface 108, and the memory 110. In some embodiments, components of portable device 100 can be integrated: for example, memory 110 and controller 112 could be integrated and provided in or on the same physical component. In some instances, portable devices can include multiple controllers or processors (for example, either or both of optical imaging device 106 and wireless communication interface 108 could include a purpose-specific dedicated processor), and in the present disclosure, all controllers/processors of portable device may be collectively referred to as controller 112.

Portable device 100 can be configured and programmed to run application software that can provide remote control functionality for medical device 200. As used throughout this disclosure, the term “remote control” is intended to include any device combinations that operate at distances from each other, regardless of magnitudes of those distances unless otherwise specified. Such remote control application software can provide any suitable degree of control of medical device 200. In some embodiments, the control provided by a remote control application can be a reduced set of controls relative to the controls available to a user interacting directly with the medical device 200 via the device's built-in user interface. In some other embodiments, the degree of control provided by a remote control application can be essentially the same as that available to a user interacting directly with the medical device 200. In still other embodiments, the remote control application could provide a greater degree of control of the medical device 200 than available to a user directly interfacing with the medical device.

Remote control application software for portable device 100 can, in some embodiments, be stored in memory 110 and executed by controller 112. In some other embodiments, information system 50 such as a server can run remote control software that can be presented on the user interface of portable device 100 via, for example, a web browser or other client software executed by controller 112 of the portable device.

Medical device 200 can include a built-in user interface that can include hardware controls 202 such as buttons or knobs and the like, and a display 204, which may be based upon or rely upon any suitable dynamic display technology such as the aforementioned LCD, OLED, EPD, and the like. Medical device 200 can include a communication interface 208, a memory 210, and a controller 212, which may include multiple controllers/processors. The controller 212 can be operatively coupled to any or all, or any subgrouping of, the user interface (for example, hardware controls 202 and/or display 204), the communication interface 208, and the memory 210. In some embodiments, components of portable device 200 can be integrated.

Medical device 200 can include any appropriate function-specific hardware 214 to provide its intended functions as a medical device. In some examples, medical device 200 can be an infusion pump, and function-specific hardware 214 can include any or all of a pump mechanism, valves, motors, drive trains, gears, couplings, sensors, application-specific integrated circuits (ASICs), firmware, and any other accessories and appropriate components. In other examples, medical device 200 can be a patient-monitoring apparatus such as a blood pressure monitor; and function specific hardware 214 could include a pressure cuff, hose, air pump, sensors, ASICs, firmware, and any other appropriate components.

In systems and methods of the present disclosure, one or more codes 220 can be displayed on display 204 of medical device 200 and imaged by optical imaging device 106 of portable device 100. “Imaged” by the optical imaging device 106 can mean collecting (e.g., measuring and recording) sufficient information from the displayed code that information encoded in the code is able to be decoded, for example by the controller 112 of the portable device. In this regard, display 204 of medical device 200 and optical imaging device 106 of portable device 100 can be specified, chosen, selected, tested, verified, and/or validated to be compatible with the function of propagating information optically from the medical device to the portable device. Display 204 can be capable of displaying a specified type of code, such as a 2D barcode, and optical imaging device 106 can be capable of imaging the code. For example, the display 204 can be large enough and have a sufficiently high spatial resolution in order to be able to display a type of code being employed, and the optical imaging device 106 can have optical components and a sensor capable of resolving sufficient spatial detail to record an image from which the code can be decoded. In some embodiments, color could be used to encode information, in which case display 204 could be a color display, and optical imaging device 106 could be a color camera, the display and camera being capable of displaying and resolving, respectively, colors being employed in the code. In some embodiments, other optical attributes of the display-imaging device pair may be exploited, such as contrast, reflectivity, polarization, and/or any other relevant characteristics.

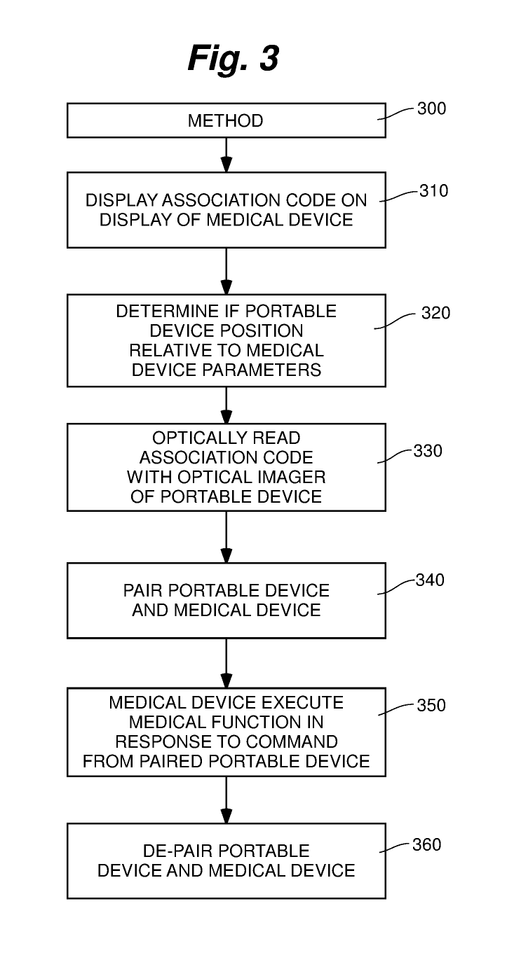

FIG. 3 is a flow diagram of an illustrative example of a method 300 for pairing a portable device with a medical device. The portable device can be the same as, or similar to, for example, portable device 100 of FIGS. 1 and 2, and the medical device can be the same as, or similar to, for example, medical device 200 of FIG. 2, which could be the same as, or similar to any of medical devices 1-8 of FIG. 1. Method 300 can be performed with a medical system the same as, or similar to the system of FIG. 1, in any of the possible compatible variations contemplated in the present disclosure. Method 300 does not necessarily require all elements shown in the flow diagram of FIG. 3. Method 300 also can include other elements not shown in FIG. 3.

Method 300 can include, at 310, displaying an association code, which may be represented by example code 220, on dynamic display 204 of a medical device 200. The association code can be displayed in any suitable machine-readable code format, or combination of code formats, that can be displayed on display 204, read by optical imaging device 106, and decoded by controller 112 of portable device 100. Possible code formats that can be used include, but are not limited to, alphanumeric text, one-dimensional (1D) barcodes such as sequences of lines of varying thickness, two-dimensional (2D) barcodes such as Quick Response (QR) codes and other codes that can include matrices of squares, triangles, hexagons and/or other shapes, and other code formats that may currently exist or be introduced in the future. (Note that in some nomenclatures, what is referred-to in the present disclosure as a “2D” barcode may be referred to as a “3D” barcode, and what is referred-to in the present disclosure as a “1D” barcode may be referred to as a “2D” barcode.) Possible code formats could include color to encode information as well as density patterns, and could vary in appearance over time (for example, as a sequence of frames, a motion, a blinking pattern, an animation, or as a movie, etc.). In some examples, a code format that does not rely solely on characters nor symbols of a natural human language can be employed. In some examples, a code format that does not include any characters nor symbols of a natural human language can be employed. Code formats can be selected and used based on a variety of attributes, such as information density, difficulty in spoofing or otherwise compromising, robustness, ease of machine readability, or any other appropriate property.

The association code can include any suitable association information. Association information, as used herein, can refer to any information included in the association code, regardless of whether the information is particularly used for an “association” purpose. Association information can include medical device identification information, which can be any of a Unique Device Identifier (UDI), a manufacturer specific identification code and/or serial number, a communication network identification code or address (for example, a media access control (MAC) address), a hospital- or other organization-specific identifier, or any other suitable identification information. Association information can include information about one or more expected or preferred communication channel(s) or mode(s) for future communication (e.g., Bluetooth, via hospital infrastructure WiFi, via a specified server, via a peer-to-peer WiFi network, etc.)

Association information can include information identifying the model of the medical device and/or any suitable information (e.g., parameters) about the configuration or status, whether permanent or transient, of the medical device. Association information can include date, time, and location information, and the like. Association information can include information about a specific medical function that the medical device is configured or intended to perform. For example, in the case of an infusion pump, such information could include information about a particular infusion, such as a medicament, a dose or rate of dosage, syringe parameters, an amount of medicament available for delivery, an amount of medicament already delivered, and so on. Association information can include patient specific information such as weight, height, age, and so on, and can also include patient identification information.

The controller 212 of medical device 200 can be configured and programmed to calculate, compile, and/or otherwise provide the association information, and can be configured and programmed to encode the association information into the association code. In some embodiments, another entity (such as information system 50) can contribute association information and/or the association code, in whole or in part, to medical device 200 for display on dynamic display 204.

Generally, an association code can include association information that is time-sensitive, transient, or perishable, and that can be verified in other parts of Method 300 before a pairing is completed. The perishable nature of association codes can provide an important security feature, making them more difficult to spoof or otherwise compromise. In comparison, a permanent or infrequently-changing code might be vulnerable to being inappropriately duplicated, with, for example, a still image or other copy of the duplicated code being presented to an optical imaging device in place of a legitimate code actually displayed on dynamic display 204 of medical device 200.

Method 300 can include, at 320, determining whether the portable device 100 is positioned relative to the medical device 200 within a predetermined range of positioning parameters. Aspects of this determination are discussed in further detail elsewhere herein. Determining that the portable device 100 is positioned relative to the medical device 200 within the predetermined range of positioning parameters can be referred-to herein as a positive result. Alternately, finding that the portable device 100 is not positioned relative to the medical device 200 within the predetermined range of positioning parameters can be referred-to herein as a negative result.

At 330, the method can include optically reading the association code from the dynamic display 204 of medical device 200 with optical imaging device 106 of portable device 100. Optically reading a code can include imaging the code with sufficient image quality (for example, in terms of contrast, focus, distortion, etc.) such that information encoded in the code can be decoded. Optically reading a code can also include the decoding of the information from an image captured by the optical imaging device 106.

The determination 320 of whether the portable device 100 is positioned relative to the medical device 200 within a predetermined range of positioning parameters can be performed as a safety measure to try to ensure that the portable device is oriented or aimed (or that the optical imaging device 106 of portable device is aimed) at medical device 200, and not at another medical device that also may be displaying an association code, so that the association code optically read at 330 can be regarded with confidence as belonging to the medical device with which the user desires and intends to pair the portable device. The predetermined range of positioning parameters can be defined (as detailed further herein) to reduce the possibility of the portable device 100 optically reading (at 330) and/or processing a code other than from a medical device at which the portable device is deliberately aimed. In some embodiments, having a clear line-of-sight between optical imaging device 106 and an association code may be necessary for the portable device 100 to be positioned relative to the medical device 200 within the predetermined range of positioning parameters. However, in some embodiments, the existence of a clear line-of-sight may not by itself be sufficient for the portable device 100 to be positioned relative to the medical device 200 within the predetermined range of positioning parameters; in these embodiments, additional more stringent positioning conditions may need to be met in addition to having a clear line-of-sight.

In various embodiments of method 300, determining at 320 and optically reading at 330 can be interrelated in various ways which may not necessarily be illustrated in FIG. 3. For example, in some embodiments, the determination 320 can be performed before the optical reading 330, and in other embodiments, the optical reading 330 can be performed before the determination 320. As an example of the latter, the optical reading 330 can be performed by the portable device 100 with optical imaging device 106, and an image of the association code captured or recorded as part of the optically reading can be analyzed (as discussed further herein) as part of the determination 320. (If the determination 320 has a negative result in such an example, the information from the preceding optical reading 330 might then be discarded.) As an example of the former, if the determination 320 is performed first and produces a positive result, the method can proceed to optically reading at 330, but if the result is negative, the optical reading 330 can be delayed until another determination 330 yields a positive result, or in some cases, not performed. In some embodiments, optically reading the association code at 330 is only performed if it is determined at 320 that the portable device 100 is positioned relative to the medical device 200 within a predetermined range of positioning parameters. In some embodiments, a positive determination at 320 is a sufficient condition for optically reading the association code at 330, but is not necessarily a prerequisite. In some embodiments, determination of negative and/or positive results may be communicated to the user of device 100, such as via a suitable message on display 104.

Another way in which determining at 320 and optically reading at 330 can be interrelated is illustrated as follows. In some embodiments, optically reading the association code at 330 can be considered to have two or more parts, including capturing an image of the association code, and then decoding the association code after the image has been captured. The determination 320 could be performed by analysis of the image captured of the association code, but the decoding part of the optically reading 330 might not be performed subsequently if the determination has a negative result.

The determination 320 of whether the portable device 100 is positioned relative to the medical device 200 within a predetermined range of positioning parameters can be performed by any suitable processor. In some cases, the determination 320 can be performed by the controller 112 of the portable device 100, for example, as a component of remote control application software being executed by the controller. In some embodiments, the determination 320 can be performed by the controller 212 of the medical device 200. In some embodiments, the determination 320 can be performed by a processor that is external to both the portable device 100 and the medical device 200, such as computing or other information system 50 of FIG. 1. Some embodiments of method 300 can include communicating from the portable device 100 information related to the association code read by the portable device, via a wireless communication mode. If the determination 320 is performed by a processor external to portable device 100, communicated information related to the association code read by the portable device can include information relevant to the determination, such as an image of the association code captured by the optical imaging device 106.

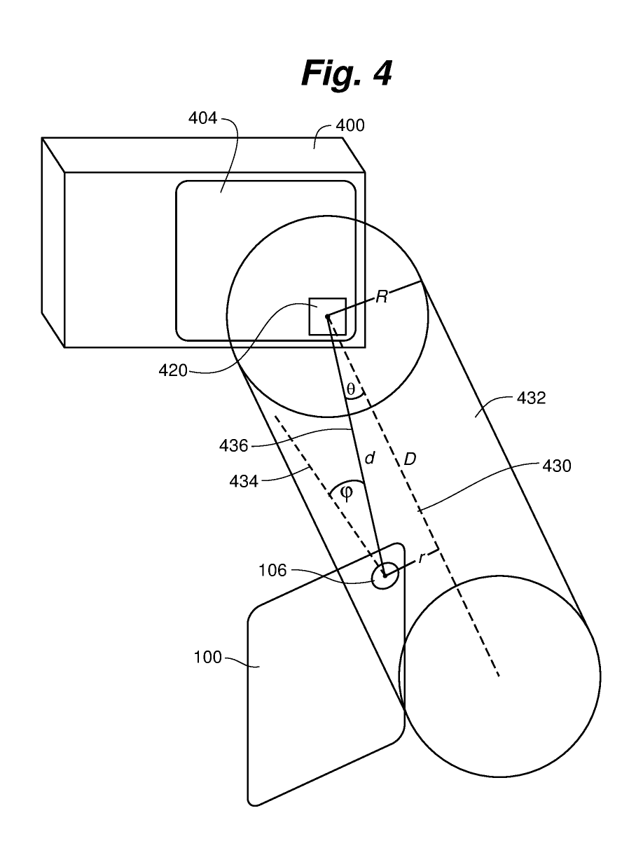

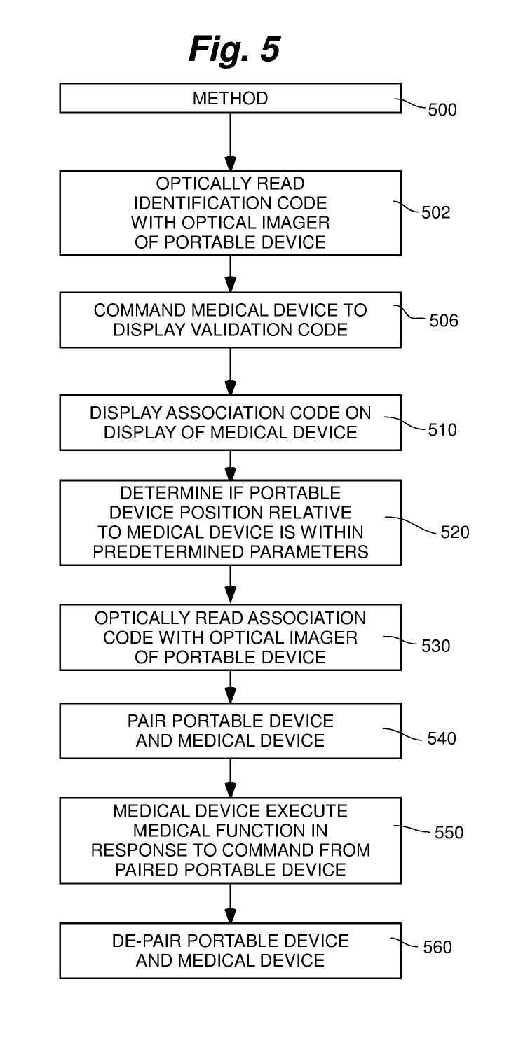

The predetermined range of positioning parameters can be defined, described, or specified in any appropriate way. FIGS. 4 and 5 schematically illustrate some examples of possible positioning parameters, or quantities related to positioning parameters, that can be used. FIG. 4 is a schematic illustration of relative positioning between a medical device 400, which can be the same as, or similar to any of medical devices 1-8 and 200, and portable device 100. Medical device 400 is illustrated as displaying a code 420 on a dynamic display 404. A line segment/axis 430 can be defined, extending away from the code 420 generally perpendicularly to the display 404 and having a length D. A cylindrical volume of space 432 can be defined about line segment/axis 430, extending out to a radius R. Positioning parameters can be defined, and then a range of positioning parameters can be predetermined, within which the portable device 100 or part of the portable device such as the optical imaging device 106 are positioned within the cylindrical volume 432. This is just one way to describe relative positioning between a portable device and a medical device. A variation on the parameters defining cylindrical volume 432 could be to specify that the distance d of a line segment 436 between the code 420 and the optical imaging device 106 is less than a predetermined value, such as, for example, 10 cm, 20 cm, 30 cm, 40 cm, 50 cm, 60 cm, or any other suitable value, when the portable device 100 is positioned relative to the medical device 400 within a predetermined range of positioning parameters. Such a distance parameter d could be applied in combination with a radial distance r between the optical imaging device and the line segment/axis 430 being within a predetermined range or value, or an angle θ between line segments 436 and 430 being within a predetermined range or value. For example, r could be required to be within a predetermined distance value such as 3 cm, 5 cm, 7 cm, 10 cm, or any other suitable value, and/or θ could be required to be within 20 degrees, 15 degrees, 10 degrees, 5 degrees, or any other suitable value. Another possible positioning parameter could be an angle φ between an optical axis 434 of the optical imaging device 106 and line segment 436, essentially quantifying how closely the optical imaging device is aimed toward the code on the display 404. Yet another possible positioning parameter could be an angle (not illustrated) quantifying rotation of the portable device 100 relative to the code 420 and pump 400 about line segment 436; that is, how closely are the portable device and medical device both oriented “up,” as opposed to “sideways” or “upside down” relative to each other. These are just some examples, and the predetermined range of positioning parameters can be defined, described, or specified in any appropriate way. While some of the parameters or variables illustrated in FIG. 4 can be used to specify, for example, substantially cylindrical or conical volumes of space in which it may be deemed necessary or desirable for a portable device to be located, it is anticipated that any arbitrarily-shaped volume(s) of space could be defined with appropriate ranges of positioning parameters. Any suitable way of defining positioning parameters, and any suitable predetermined range of positioning parameters can be defined and used to reduce the possibility of the portable device 100 optically reading (at 330) and/or processing a code other than from a medical device at which the portable device is deliberately aimed.

Determination of whether the portable device 100 is positioned relative to the medical device 200 within a predetermined range of positioning parameters can be performed in any suitable manner, and with any appropriate hardware. It is anticipated that the determination can be performed with the optical imaging device 106 of portable device 100, but other implementations are possible. Any suitable information measured or obtained by optical imaging device 106 can be used in the determination. For example, if optical imaging device 106 has an adjustable focus mechanism, information from the focus mechanism could be used, such as a range determination or estimation performed by the focus mechanism. In addition or alternatively, image analysis could be performed on one or more images captured by the optical imaging device 106. With a target having a known shape and size (for example, a displayed code, the medical device 200 as a whole, and/or some other fiducial marker(s) of the medical device), and knowledge of the properties of the optical imaging device 106, analysis of an image could yield a distance between the optical imaging device and the target, and furthermore, consideration of image properties such as perspective distortion could yield a relative angular relationship between the two.

In general, determining relative positioning can depend upon information about the capabilities of the specific hardware involved. For example, for an optical imaging device that is a camera, lens characteristics (for example, focal length(s), focus mechanism calibration, aberrations, etc.) and sensor characteristics (array size, resolution, etc.) might be used. Given that the systems and methods of the present disclosure are intended to be practicable with a variety of suitable hardware, the predetermined range of positioning parameters can be defined independently of any particular hardware capabilities of any particular portable device. Accordingly, it is anticipated that different portable devices having different hardware can be capable of determining whether they are positioned relative to medical devices within the predetermined range of positioning parameters, with the parameters being identically described for the different devices, and with the processor performing the determination being capable of interpreting the positioning parameters in light of the specific hardware capabilities being used to measure the positioning of the portable device relative to the medical device.

Method 300 can include at 340, pairing the portable device 100 and the medical device 200, if predetermined conditions for entering paired communication are met. Any suitable conditions can be used to initiate entering paired communications. When paired, the portable device 100 and medical device 200 can be communicatively linked such that the medical device responds to commands sent from the portable device to the medical device. Commands sent from portable device 100 to medical device 200 can be specifically addressed only to the medical device. Any appropriate communication infrastructure 12 can be used for paired communication. A paired link between portable device 100 and medical device 200 can include a secure and/or encrypted communication link between the devices. A paired communication link between devices can include any other suitable characteristics that may be desired for inter-device communication.

In some embodiments, paired communication is entered at 340 only if, at 320, it is determined that the portable device 100 is positioned relative to the medical device 200 within the predetermined range of positioning parameters. However, this criterion is not necessarily required in all cases. In some other embodiments, paired communication can be entered at 340 even if the determination at 320 does not find that the portable device 100 is positioned relative to the medical device 200 within the predetermined range of positioning parameters. In some applications, performing the determination at 320 may not be required. This may be suitable in applications where there is unlikely to be more than one medical device that might be able to pair with a portable device. One such application could be a home care scenario, where a patient receives therapy from a single medical device (such as, but not limited to, an infusion pump) that can be controlled by a paired portable device.

In some embodiments, paired communication is entered at 340 only if, at 330, the association code is optically read from the dynamic display 204 of the medical device 200 with the portable device 100. In some embodiments, paired communication is entered at 340 only if both (a) it is determined (at 320) that the portable device 100 is positioned relative to the medical device 200 within the predetermined range of positioning parameters, and (b) the association code is optically read from the dynamic display 204 of the medical device with the portable device.

In some embodiments, if the portable device is not positioned relative to the medical device within the predetermined range of positioning parameters, then the method 300 includes not pairing the portable device and the medical device, or preventing or blocking the portable device and medical device from being paired.

Method 300 can include preconditions for entering paired communication at 340 other than a positive result from the determination at 320 and/or optically reading the association code at 330. The preconditions can include appropriateness for entering into paired communication and/or control, as defined in any appropriate way and as ascertained in any suitable manner. Appropriateness for entering into paired communication can be an attribute of either or both of the medical device 200 and portable device 100. As an example, a medical device 200 that is an infusion pump could be appropriately paired to a portable device 100 for remote control from the portable device in only some infusion applications, but not others. In another example, a portable device 100 could be subject to appropriateness criteria related to whether the portable device is executing other processes that could potentially interfere with the portable device performing as a paired remote control for medical device 200. Whether appropriateness preconditions are met could be determined by any suitable processor. In some embodiments, a user or other agent could manually specify appropriateness for pairing of a medical device 200, a portable device 100, or any combination of medical device(s) and portable device(s).

Preconditions for entering paired communication at 340 can include any suitable preconditions relating to the association information of the association code optically read at 330. Preconditions could include whether a date, time, and or location of association information are consistent with the same parameters of the portable device 100, or of information system 50. Preconditions could include whether association information relating to the specific medical function that the medical device is to perform and/or patient specific information agree with the same kind(s) of information already resident in the memory 110 of portable device 100, and/or in information system 50, which could be, for example, a hospital information system. In some embodiments, optical imaging device 106 could be used to read information from objects such as an identification wristband of a patient, a medicament container, and so on, with verification of such information potentially being used as a precondition for entering paired communication between the portable device 100 and medical device 200, and/or for other confirmation relating to operation of the medical device.

Preconditions for entering paired communication at 340 could include consideration of aspects of communications infrastructure 12, such as whether appropriately secure and reliable communication links are established between the portable device 100 and medical device 200, and possibly information system 50.

Pairing at 340 of Method 300 of the portable device 100 with the medical device 200 can be commanded by any suitable processor. A processor commanding or otherwise initiating pairing can be configured to determine whether predetermined conditions for entering paired communication have been met, and if so, it can issue commands to any or all of portable device 100, medical device 200, and/or information system 50, that can result in establishment of a paired communication link between the portable device and the medical device.

In some embodiments, the controller 112 of the portable device 100 is programmed and configured, if the association code is read from the dynamic display 204 of the medical device 200, to determine whether predetermined conditions for entering paired communication with the medical device have been met, including considering information related to the association code. If the predetermined conditions for entering paired communication with the medical device 200 are determined to have been met, the controller 112 of the portable device can be programmed and configured to communicate, via the wireless communication interface 108, a command to the controller 212 of the medical device to pair with the portable device 100. The controller 212 of the medical device 200 can be programmed and configured to pair with the portable device 100 under the (communicated) command of the controller 112 of the portable device 100. Optionally, the controller 112 of portable device 100 could also communicate with information system 50 to, for example, inform the information system 50 of the pairing of the portable device with the medical device 200, or to command the information system 50 to act as an intermediary or other participant in the pairing. In some of these embodiments, commands or other communications from the controller 112 of the portable device 100 can include information related to the association code read by the optical imaging device 106.

In some embodiments, the controller 212 of the medical device 200 can be programmed and configured to pair with the portable device 100 after the controller of the medical device has determined that predetermined conditions for entering paired communication with the portable device have been met. Such a determination by the controller 212 of the medical device 200 can include consideration of information related to the association code communicated by the controller 112 of the portable device via its wireless communication interface 108 after having been read by the optical imaging device 106. A determination by the controller 212 of the medical device 200 of whether to pair the portable device with the medical device can include any relevant considerations pertinent to pairing discussed herein, including, for example, determining whether the portable device is positioned relative to the medical device within a predetermined range of positioning parameters, although this is not required; the relative position determination can be performed, in some cases, by the controller of the portable device. A determination by the controller 212 of the medical device 200 of whether to pair can be made alternatively to such a determination being made by the controller 112 of the portable device 100. In some embodiments, a determination by the controller 212 of the medical device 200 of whether to pair the portable device with the medical device can be made in addition to such a determination being made by the controller 112 of the portable device 100. In some embodiments, both controllers 112 and 212 of the portable device 100 and medical device 200 could be required to determine that predetermined conditions for entering paired communication with each other have been met before such a pairing would be entered.

An information system 50 external to the portable device 100 and medical device 200 can command pairing in some embodiments. The information system 50 can be any suitable system, residing in any suitable location. The information system 50 can include, for example, an electronic medical record (“EMR”) system, an electronic medical administration record (“EMAR”) system, a Hospital Information System (“HIS”), a pairing server or a pairing process running on a server provided specifically to perform pairing-related computing tasks, a general purpose personal computer, or another appropriate computing system. In such an embodiment involving an external information system 50, the portable device 100 can read an association code from the dynamic display 204 of the medical device 200 with its optical imaging device 106, and communicate, via its wireless communication interface 108, information related to the association code read by the optical imaging device. The information related to the association code can be any suitable information, taking any suitable form. In some embodiments, the related information can include, in decoded form, all or part of the association information encoded in the association code. In some embodiments, the related information can include information deriving or transformed from association information. In some embodiments, the related information can include an image of the association code captured by the optical imaging device 106.

The external information system 50 can be programmed and configured to receive from the portable device 100 information related to the association code read by the optical imaging device 106, and determine whether predetermined conditions for entering paired communication between the medical device 200 and the portable device have been met. If predetermined conditions for entering paired communication between the medical device 200 and the portable device 100 are determined to have been met, information system 50 can be programmed and configured to communicate commands to the controller 212 of the medical device and the controller 112 of the portable device to pair. The determination by the information system 50 whether predetermined conditions for entering paired communication between the medical device 200 and the portable device 100 have been met can include any relevant considerations discussed herein, including, for example, determining whether the portable device is positioned relative to the medical device within a predetermined range of positioning parameters, although this is not required; the relative position determination can be performed, in some cases, by the controller of the portable device. A determination by an external information system 50 whether to pair can be made alternatively or in addition to such a determination being made by either or both of the controller 112 of the portable device 100 and/or the controller 212 of the medical device 200.

In some embodiments, determining whether predetermined conditions for entering paired communication have been met can be a shared task among multiple processors. As an example, in one scenario (1) a processor of a medical device could apply appropriateness criteria to determine whether the medical device is in a state in which pairing is appropriate; (2) a processor of a portable device could determine whether the portable device is positioned relative to the medical device within a predetermined range of positioning parameters; and (3) a processor of an external information system could consider the outcomes of the determinations of (1) and (2), as well as its own determinations of whether other predetermined conditions are met, before the processor of the external information system potentially issues commands to initiate paired communication between the portable device and the medical device.

In some embodiments, pairing the portable device and the medical device includes receiving confirmation from a user to make the pairing, but this is not necessary in all embodiments. Any suitable user confirmation protocol can be used. Some user confirmation protocols can include information displayed on either or both of medical device 200 and portable device 100. In some examples, displayed information on both medical device 200 and portable device 100 could be coordinated to increase the likelihood that a user would recognize that the two devices are acting/communicating in a paired manner. A user confirmation protocol could include a required response from a user to the user interface either or both of medical device 200 and portable device 100. Such a response or responses could include a touch, an audible response, a motion, or any other detectable action. In some examples, receiving confirmation from a user does not require physical contact with the medical device, as might be desirable to avoid contamination.

When paired, the portable device 100 can act as a remote control unit for the medical device 200 and can send commands to the medical device. At 350 of Method 300, the medical device 200 can execute a medical function in response to a command sent from the portable device 100.

After pairing at 340 of Method 300, portable device 100 and medical device 200 can remain paired for any suitable duration of time. At 360 of Method 300, the portable device 100 and medical device 200 can be de-paired. As used in this disclosure, the term “de-pair” refers to an action in which a paired communication link is paused, suspended, terminated, or otherwise stopped. De-pairing can be triggered by any suitable criteria. Some possible de-pairing criteria can include the completion of a medical procedure, such as a medicament delivery by an infusion pump. De-pairing can be manually commanded by a user. In some examples, a medical system can include one or more location systems configured and capable of determining one or more location, such as a location of portable device 100 and one or more of medical devices 1-8 and/or 200. Location systems can use any suitable technology, such as global positioning system (GPS) and/or other satellite navigation systems, radio-frequency identification (RFID), WiFi-assisted location, inertial dead-reckoning, and so on. In some embodiment, if the location of the portable device 100 and/or medical device 200 meets one or more pre-determined conditions, the devices can be de-paired. De-pairing can be commanded by any suitable processor, such as controller 112 of portable device 100, controller 212 of medical device 200, and/or information system 50.

Following de-pairing, previously-paired portable device 100 and medical device 200 can subsequently be re-paired to reestablish paired communication. In some embodiments, re-pairing devices that had been paired previously can be initiated or commanded after conditions for re-pairing have been satisfied. In some cases, such conditions for re-pairing can differ from preconditions for pairing that are to be satisfied for devices that do not have a recent or relevant history of having been paired, and could be less stringent conditions. In some embodiments, devices can be de-paired in a manner that requires a subsequent pairing to satisfy preconditions no less stringent than for a pairing of devices not previously paired.

Other methods for pairing a portable device with a medical device are contemplated in the present disclosure. FIG. 5 is a flow diagram of another illustrative example of a method 500 for pairing a portable device with a medical device. Method 500 can include multiple elements that are similar to or the same as corresponding elements of Method 300 of FIG. 3. Elements 510, 520, 530, 540, 550, and 560 of Method 500 can be substantially the same as or similar to elements 310, 320, 330, 340, 350, and 360 of Method 300, to the extent that the descriptions of the elements of Method 300 are not inconsistent with specific descriptions of Method 500.

At 502, Method 500 can include optically reading, with optical imaging device 106 of portable device 100, an identification code that identifies medical device 200. The identification code can be displayed in any suitable machine-readable code format, or combination of code formats, similarly as for the association code, and the discussion of possible code formats provided in relation to the association code generally applies to code formats for the identification code.

The identification code can include any suitable identification information. Identification information, as used herein, can refer to any information included in the identification code, regardless of whether the information is particularly used for an “identification” purpose. The identification information can include any suitable information that uniquely references a specific individual medical device, such as a Unique Device Identifier (UDI), a manufacturer specific identification code and/or serial number, a communication network identification code or address, a hospital- or other organization-specific identifier, or any other suitable identification information. The identification information of the identification code can include sufficient information to allow portable device 100 in possession of the identification information to communicate with a specific individual medical device referenced by the identification code. In some examples, this information can include a unique network address of the specific individual medical device that allows portable device 100 to establish a communicative connection over communication infrastructure 12 to the medical device. In some other examples, the identification information of the identification code can include information that, when presented by the portable device 100 to an external system such as information system 50, allows the information system to enable a communicative connection between the portable device and the specific medical device referenced by the identification code. In one such example, the identification information of the identification code includes information that identifies a specific individual medical device, but not a network address for the medical device. In this example, the information system 50 possesses the network address for the medical device and facilitates communication between portable device 100 and medical device 200 after receiving identification information communicated by the portable device after the portable device read said identification information via the identification code.

The identification code that is optically read at 502 of Method 500 can be displayed in any suitable location. In some examples, the identification code is displayed on the medical device 200. In some instances, the identification code is displayed on dynamic display 204 of medical device 200. The identification code can, in some instances, include less encoded information than an association code, and accordingly, it can occupy a smaller portion of the display area of dynamic display 204 than an association code. In some embodiments, the identification code can be displayed on dynamic display 204 for a substantially greater fraction of time that medical device 200 is powered, as compared with a fraction of time that an association code might be displayed. In some embodiments, the identification code can be displayed on dynamic display 204 whenever the medical device is in a state in which it can be paired or a state in which it can commence pairing. In some embodiments, the identification code can be, substantially, permanently displayed on an enclosure, housing, or case of medical device 200, as for example, being printed on a surface thereof. In some embodiments, the identification code can be non-permanently affixed to a medical device, such as with an adhesive sticker. In some cases, the identification code can be provided spatially apart from a medical device, such as on a printed sheet or on a workstation display screen.

In some embodiments, optically reading the identification code at 502 can be performed in conjunction with determining whether the portable device 100 is positioned relative to the medical device 200 within a predetermined range of positioning parameters. Such a determination can be performed specifically relating to optically reading the identification code at 502, and independently from a determination (at 520) performed in conjunction with reading an association code (at 530). The same or different predetermined ranges of positioning parameters can be used for separate determinations. Similarly as with 320 and 330 of Method 300, optically reading the identification code at 502, or using the information from such a reading, can depend upon whether an associated positioning determination has a positive or negative result.

In some embodiments, a single determination (for example, at 520) of whether portable device 100 is positioned relative to medical device 200 within predetermined parameters can be performed in conjunction with both optically reading the identification code at 502 and optically reading the association barcode at 530. Such a single determination may be appropriate or acceptable if a time interval between the two readings is sufficiently small that the relative positions of the portable device 100 and medical device 200 are not likely to change significantly between readings.

At 506, Method 500 can include commanding the medical device 200 to display an association code on dynamic display 204 of the medical device 200. In some embodiments, commanding at 506 is performed in response to reading the identification code at 502, and such a command can be issued by any appropriate device, including processor 112 of the portable device 100. In some examples, the portable device 100 can communicate information related to the identification code read by the portable device (for example, via a wireless communication mode), and a processor external to the portable device (for example, of information system 50) can receive the information related to the identification code and in response, command the medical device 200 to display the association code. In some embodiments, the medical device 200 can be commanded at 506 to display an association code on dynamic display 204 in response to events other than reading an identification code at 502. For example, a user could initiate a pairing by interacting in any suitable manner with the user interface of either the medical device 200 or the portable device 100, which could result in a command to the medical device to display an association code on dynamic display 204. In another example, information from a location system that portable device 100 is within a specified proximity of medical device 200 could lead to a command to the medical device 200 to display an association code.

After a command to display an association code is issued at 506, Method 500 can proceed at 510 to displaying the association code on dynamic display 204 of medical device 200. As stated elsewhere, elements 510, 520, 530, 540, 550, and 560 of Method 500 can be substantially the same as or similar to elements 310, 320, 330, 340, 350, and 360 of Method 300.

Multiple medical devices can be paired with a portable device with methods of the present disclosure. Variations on Methods 300 and 500 can include repeating any of the elements of the methods to pair a second or further medical device(s) with portable device 100 such that the second or further medical device(s) respond(s) to commands sent from the portable device to the device(s).

In some embodiments, members of a group of medical devices can be associated with each other before commencement of a pairing process. Such associations can be defined or established in any suitable manner, and information about such associations can be stored or maintained in any suitable repository, such as information system 50. In some embodiments of pairing methods, multiple medical devices that are associated prior to commencement of a pairing process can be paired to a portable device after an association code is optically read from a single one of the medical devices by the portable device. In some embodiments, pairing of multiple medical devices to a portable device might proceed after association codes are read from a subset or all of the associated multiple medical devices.

As described elsewhere in conjunction with FIG. 1, in some examples multiple medical devices such as devices 1-8 can be automatically associated when mounted to an equipment rack or other support structure such as structure 10 and connected to networking hardware of the structure. In some of these scenarios, a variation on Method 500 can be practiced to pair portable device 100 with some or all of medical devices 1-8. Support structure 10 can include an identification code 11 including information identifying the support structure and by extension, any of medical devices 1-8 that are associated with the support structure (the devices being associated with the structure, for example, by virtue of being connected to networking hardware of the structure). Optically reading identification code 11 with portable device 100 can trigger any suitable processor (for example, controller 112 of portable device 100, controller 212 of medical device 200, information system 50, etc.) to proceed with a process to pair any or all of associated devices 1-8 to portable devices, as may be appropriate. Such a variation on Method 500 could include displaying an association code on one or more of the medical devices 1-8 to be paired, with pairing being achieved after the association code(s) is/are read and any other predetermined conditions for entering pairing are met. In some variations, only a single association code need be read from one of medical devices 1-8 to pair a plurality of devices to portable device 100. In some variations, an association code is read from each of the medical devices 1-8 to be paired to portable device 100.

In some embodiments, more than one portable device can be paired or linked to the same medical device or devices. In such arrangements, information could be synchronized such that any of the multiple portable devices would display essentially the same information with regard to a linked medical device.

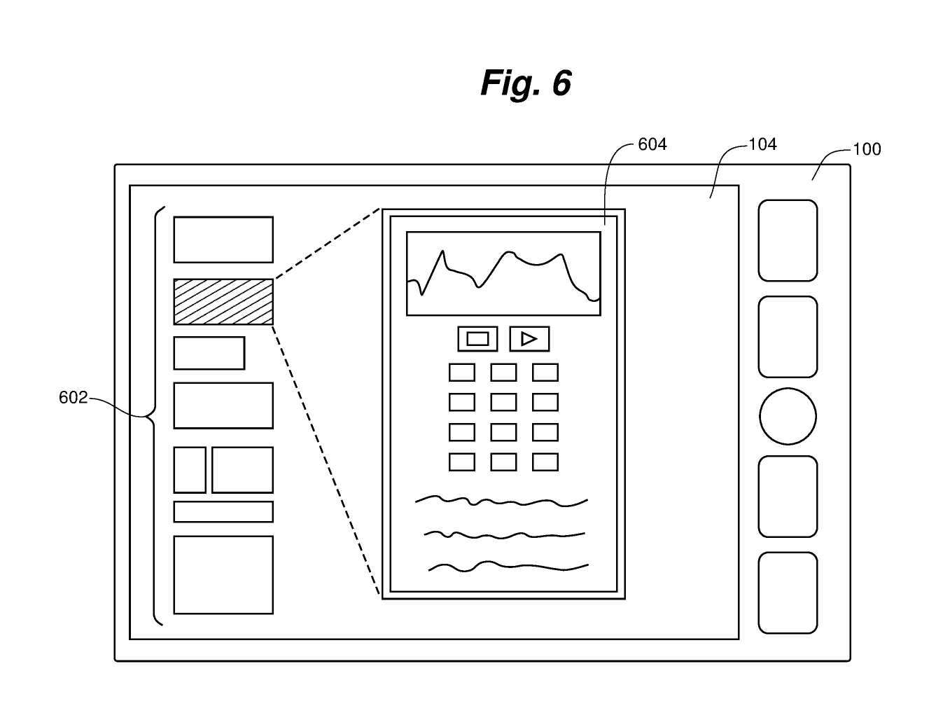

In some embodiments where multiple medical devices are paired with portable device 100, methods of the present disclosure can include rendering on display 104 of the portable device a virtual representation of the paired medical devices that emulates the real-world spatial relationship between the medical devices. FIG. 6 is a schematic representation of portable device 100 with a simulated virtual representation 602 of medical devices 1-8 of FIG. 1 on display 104, as well as a simulated virtual control panel 604. One of the virtual medical devices of virtual representation 602 is shown with hatched lines, indicating the device to which virtual control panel 604 corresponds. This is merely an example of possible user interface features. Information about the real-world spatial relationship between the medical devices, to inform the virtual representation of the devices, can be garnered in any suitable manner. In some examples, spatial information about medical device positioning could be manually entered via a user interface. In configurations where medical devices 1-8 are associated with structure 10 by virtue of being connected to networking hardware of the structure, information about the real-world spatial relationship between the medical devices could be inferred or otherwise established from the connections to the networking hardware (for example, particular hardwired network ports could be associated with specific physical locations of medical devices relative to structure 10). In some examples, images and/or other information collected by optical imaging device 106 and/or other sensors could be interpreted to determine the relative positions of medical devices.

In some embodiments, portable device 100 can display on display 104, in the manner of a camera viewfinder, real-time images being captured by optical imaging device 106 before and/or while reading identification and/or association codes, and/or after one or more pairings have been established. Such a display could assist a user in comparing/verifying what the optical imaging device is imaging (“seeing”) in relation to what the user can see of the actual medical device(s) with the user's own eyes. Such an electronic viewfinder display on display 104 of the portable device 100 can be artificially enhanced (for example, with outlining, highlighting, or other visual cues) to assist the user in recognizing a medical device of interest (for example, a device whose code is being targeted/read). In some examples, the user can select a medical device for further interaction from such a real-time electronic viewfinder display, for example, by touching the image of the device on a touch-screen display. Selecting a medical device for further interaction could be followed by, for example, the selected medical device being commanded to display an association code, or a virtual control panel for the selected medical device being activated.

This disclosure is to be understood to be not limited to the particular examples described herein, but rather should be understood to cover all aspects of the disclosure and equivalents thereof. Various modifications, processes, and components, as well as numerous structures to which the disclosure can be applicable, will be readily apparent to those of skill in the art upon review of the instant specification.

1. A method for pairing a portable device with a medical device, comprising:
displaying an association code on a dynamic display of a medical device;
determining whether a portable device is positioned relative to the medical device within a predetermined range of positioning parameters;
if the portable device is positioned relative to the medical device within the predetermined range of positioning parameters, optically reading the association code from the dynamic display of the medical device with the portable device; and
only if:
(a) the portable device is positioned relative to the medical device within the predetermined range of positioning parameters, and
(b) the association code is optically read from the dynamic display of the medical device with the portable device, then:
pairing the portable device and the medical device such that the medical device responds to commands sent from the portable device to the medical device.

2. The method of claim 1, further comprising:
if (c) the portable device is not positioned relative to the medical device within the predetermined range of positioning parameters, then not pairing the portable device and the medical device.

3. The method of claim 1, wherein the predetermined range of positioning parameters are defined independently of particular hardware capabilities of the portable device.

4. The method of claim 1, wherein the predetermined range of positioning parameters define positioning a camera of the portable device within 50 cm of the association code displayed on the medical device and within 5 cm of an axis centered on the association code and normal to the association code.

5. The method of claim 1, wherein pairing the portable device and the medical device includes receiving confirmation from a user to make the pairing.

6. The method of claim 4, wherein receiving confirmation from a user does not require physical contact with the medical device.

7. The method of claim 1, further comprising communicating from the portable device information related to the association code read by the portable device, via a wireless communication mode.

8. The method of claim 6, further comprising a processor external to the portable device and the medical device receiving the information related to the association code read by the portable device, and
wherein pairing the portable device and the medical device includes the processor, in response to receiving the information related to the association code, commanding pairing of the portable device and the medical device.

9. The method of claim 1, further comprising pairing the portable device and a second medical device such that the second medical device responds to commands sent from the portable device to the second medical device.

10. The method of claim 8, further comprising rendering on a display of the portable device a virtual representation of the medical device and the second medical device that emulates a real-world spatial relationship between the medical device and the second medical device.

11. The method of claim 1, further comprising optically reading, with the portable device, an identification code that identifies the medical device, and wherein displaying the association code on the dynamic display of the medical device is performed in response to optically reading the identification code.

12. The method of claim 1, further comprising the medical device executing a medical function in response to a command sent from the portable device.

13. The method of claim 1, wherein if the portable device is not positioned relative to the medical device within the predetermined range of positioning parameters, then the association code is not optically read from the dynamic display of the medical device with the portable device.

14. A method for pairing a portable device with a medical device, comprising:
optically reading, with a portable device, an identification code that identifies a medical device;
commanding the medical device to display an association code on a dynamic display of the medical device;
determining whether the portable device is positioned relative to the medical device within a predetermined range of positioning parameters;
if the portable device is positioned relative to the medical device within the predetermined range of positioning parameters, optically reading the association code from the dynamic display of the medical device with the portable device;
only if:
(a) the portable device is positioned relative to the medical device within the predetermined range of positioning parameters, and
(b) the association code is optically read from the dynamic display of the medical device with the portable device, then:
pairing the portable device and the medical device such that the medical device responds to commands sent from the portable device to the medical device; and
if (c) the portable device is not positioned relative to the medical device within the predetermined range of positioning parameters, then not pairing the portable device and the medical device.

15. The method of claim 13, wherein commanding the medical device to display an association code on a dynamic display of the medical device is performed in response to reading the identification code.

16. The method of claim 13, wherein the identification code is displayed on the medical device.

17. The method of claim 13, wherein the association code includes information particular to a current status of the medical device.

18. The method of claim 13, wherein commanding the medical device to display an association code includes a processor of the portable device commanding the medical device to display the association code.

19. The method of claim 13, further comprising communicating from the portable device information related to the identification code read by the portable device, via a wireless communication mode, and
wherein commanding the medical device to display an association code includes a processor external to the portable device receiving the information related to the identification code read by the portable device, and in response, the processor commanding the medical device to display the association code.

20. The method of claim 13, further comprising communicating from the portable device information related to the association code read by the portable device via a wireless communication mode, and
wherein pairing the portable device and the medical device includes a processor external to the portable device receiving the information related to the association code read by the portable device, and in response, the processor pairing the portable device and the medical device.

21. A medical device system, comprising:
a medical device configured to provide at least one of a therapeutic or a patient monitoring function, the medical device including:
a display capable of displaying a machine-readable code;
a communication interface; and
a controller operatively coupled to the display and the communication interface; and
a portable device, including:
a user interface configurable to present virtual controls for the medical device to a user;
an optical imaging device;
a wireless communication interface; and
a controller operatively coupled to the user interface, the optical imaging device, and the wireless communication interface,
wherein the controller of the medical device is programmed and configured to:
display a machine-readable association code on the display of a medical device; and
pair with the portable device either under the command of the controller of the portable device or an external processor, or after the controller of the medical device has determined that predetermined conditions for entering paired communication with the portable device have been met, wherein when paired, the medical device responds to commands sent from the portable device to the medical device; and
wherein the controller of the portable device is programmed and configured to:
determine whether the portable device is positioned relative to the medical device within a predetermined range of positioning parameters; and
if the controller determines that the portable device is positioned relative to the medical device within the predetermined range of positioning parameters:
read the association code from the dynamic display of the medical device with the optical imaging device of the portable device; and
communicate, via the wireless communication interface, information related to the association code read by the optical imaging device.

22. The system of claim 20, wherein the controller of the portable device is further programmed and configured to:
if the association code is read from the dynamic display of the medical device, determine whether predetermined conditions for entering paired communication with the medical device have been met, including considering information related to the association code; and
if predetermined conditions for entering paired communication with the medical device are determined to have been met, communicate, via the wireless communication interface, a command to the controller of the medical device to pair with the portable device.

23. The system of claim 20, further comprising an external computing system separate from, and communicatively coupled with, the medical device and the portable device, wherein the external computing system is programmed and configured to:
receive from the portable device information related to the association code read by the optical imaging device;
determine whether predetermined conditions for entering paired communication between the medical device and the portable device have been met; and
if predetermined conditions for entering paired communication between the medical device and the portable device are determined to have been met, communicate commands to the controller of the medical device and the controller of the portable device to pair.
